(19) United States
(12) Patent Application Publication
(10) Pub. No.: US 2009/0130666 A1
(43) Pub. Date: May 21, 2009

(54) Cytometric system including nucleic acid sequence amplification, and method

(75) Inventor: Mark F. Oldham, Los Gatos, CA (US)

Correspondence Address: KILYK & BOWERSOX, P.L.L.C., 3925 CHAIN BRIDGE ROAD, SUITE D401, FAIRFAX, VA, 22030, US

(73) Assignee: Applera Corporation, Foster City, CA (US)

(21) Appl. No.: 11/921,114

(22) PCT Filed: May 30, 2006
(86) PCT No.: PCT/US2006/020670

§ 371 Date: Nov. 28, 2008

Related U.S. Application Data
(60) Provisional application No. 60/685,119, filed on May 27, 2005.

(51) Int. Cl. C12Q 1/68; C12M 1/00; C12M 1/34

(52) U.S. Cl. 435/6; 435/287.2; 435/286.1

(57) ABSTRACT

The invention relates to a cytometric system that can include a first station capable of selectively sorting a nucleic acid sequence-containing biological material from a biological sample, and a second station where nucleic acid of the sorted nucleic acid sequence-containing biological material, can undergo a nucleic acid amplification reaction. The system can include a conduit system adapted to facilitate transfer of a sorted biological material or processed biological material from one region or station, to another. All of the stations can be controlled by a central control system. Methods of sorting a biological material from a biological sample, and methods of amplifying nucleic acid of a sorted nucleic acid sequence-containing material, are also provided.

### CROSS REFERENCE TO RELATED APPLICATION

The present application is a National Stage of PCT/US2006/020670, filed May 30, 2006, which claims a priority benefit from earlier filed U.S. Provisional Application No. 60/685,119, filed May 27, 2005, both of which are herein incorporated by reference in their entireties.

### FIELD

The present teachings relate to cytometry devices and methods using the same.

### BACKGROUND

Cytometry devices are often used in biotechnology to sort biological materials based on the individual characteristics present in the components of the biological material. A sorted biological material can then be manually transferred to a separate machine or machines for further manipulation or evaluation. This process can be difficult when using current biotechnology techniques that require very small amounts of material and/or high throughput applications.

### SUMMARY

According to various embodiments of the present teachings, a cytometric system is provided that is capable of sorting and automatically processing a biological material from a sample. According to various embodiments, the cytometric system can sort a biological material, for example, a target type of cell, and then perform a detection reaction, for example, a nucleic acid detection reaction. In some embodiments, the detection reaction can comprise a nucleic acid amplification reaction, for example, a polymerase chain reaction (PCR), involving one or more nucleic acid sequences in the cell. In some embodiments the detection reaction can comprise two or more reactions. The cytometric system can minimize fluid loss and fluid transfer errors that could otherwise occur with manual sample transfer procedures. In some embodiments, the cytometric system can sort a biological material into homogeneous sets and can then analyze the homogenous sets.

According to various embodiments, a cytometric system is provided that comprises: a sorting station adapted to sort a nucleic acid sequence-containing biological material from a sample; a detection station, for example, an amplification station adapted to amplify at least a portion of a nucleic acid sequence to form an amplification product; and a conduit system capable of directing a nucleic acid sequence-containing biological material from the sorting station to the detection station. The sorting station can be adapted to sort the nucleic acid sequence-containing biological material by at least one of size, molecular weight, cell type, staining characteristics, reflectivity, antibody binding, electric charge, and a combination thereof. The sorting station can comprise a flow cytometry device, a surface modification device, an optoelectronic material manipulation device, an optoelectronic scanning device, a combination thereof, and the like.

According to various embodiments, the conduit system can comprise at least one pump and/or at least one valve. The conduit system can comprise at least one valved conduit, wherein the at least one valved conduit can be simultaneously in fluid communication with both the sorting station and the detection station. The system can comprise a planar substrate, wherein the conduit system comprises at least one channel formed in the planar substrate. In some embodiments, the detection station can comprise a thermal cycler, or an isothermal heater or oven. The fluid channel can be in fluid communication with one or more processing regions, for example, a sorting region, a preparation region, a purification region, an amplification region, a sequencing region, a detection region, a combination thereof, and the like.

According to various embodiments, the device can comprise at least two retainment regions, each adapted to retain a respective different nucleic acid sequence-containing biological material sorted by the sorting station, wherein the conduit system is adapted to direct nucleic acid sequence-containing biological material from each of the at least two retainment regions to the detection station, for example, to a tube for amplification and thermal cycling. Aliquots of a single sorted biological material or multiple sorted biological materials, can be contained in a single region, for example, a capillary or a tube, and can be separated from other aliquots by intervening layers of immiscible fluid. The detection station can comprise an amplification station comprising one or more reagents to enable sequence amplification, for example, polymerase chain reaction, of a nucleic acid sequence, upon thermal cycling. The amplification station can comprise at least one rotatable platen. The amplification system can comprise one or more tubes, wells, regions, recesses, or the like, to retain a respective nucleic acid sequence of a sorted biological material, during amplification.

According to various embodiments, the system can comprise a control unit operatively connected to the sorting station and to the detection station. The control unit can comprise a computer system.

According to various embodiments, the system can further comprise a preparation station capable of isolating a nucleic acid sequence from a nucleic acid sequence-containing biological material, wherein the conduit system can be adapted to direct a nucleic acid sequence-containing biological material from the sorting station to the preparation station and the conduit system can be adapted to direct an isolated nucleic acid sequence from the preparation station to the detection station. The preparation station can comprise a separation device, for example, a centrifuge, and/or the preparation station can comprise a lysis station.

According to various embodiments, the system can further comprise a sequence detection station, wherein the conduit system is capable of directing an amplified product from the amplification station to the sequence detection station. The sequence detection station can comprise, for example, at least one of a capillary electrophoresis device and a chromatographic column device.

According to various embodiments, the system can comprise a housing for containing the sorting station, the amplification station, and the conduit system.

According to various embodiments, a method is provided for processing a nucleic acid-containing sample with a system as described herein, wherein the method includes at least one cytometric separation procedure and at least one nucleic acid sequence detection procedure.

It is to be understood that both the foregoing general description and the following detailed description are exemplary and explanatory only, and are intended to provide a further explanation of the various and many embodiments described herein.

### BRIEF DESCRIPTION OF THE DRAWINGS

Various embodiments of the present teachings are exemplified in the accompanying drawings. The teachings are not limited to the embodiments depicted in the drawings, and comprise equivalent structures and methods as set forth in the following description and as would be known to those of ordinary skill in the art in view of the present teachings. In the drawings:

FIG. 1 is a block diagram of the various stations of a system according to various embodiments;

FIG. 2 is a top plan view of a system according to various embodiments;

FIG. 3 is a sectional view of a system according to various embodiments; and

FIG. 4 is a top plan view of a system according to various embodiments.

### DETAILED DESCRIPTION OF THE VARIOUS EMBODIMENTS

According to various embodiments of the present teachings, a cytometric system is provided that is capable of sorting and automatically processing a biological material from a sample. According to various embodiments, the cytometric system can sort a nucleic acid sequence-containing biological material, for example, a target type of cell, from other materials in the sample, and then perform a reaction with the nucleic acid sequence. The reaction can be a nucleic acid sequence detection reaction and/or a nucleic acid sequence amplification reaction, for example, a hybridization reaction, or a polymerase chain reaction (PCR), of one or more nucleic acid sequences in or extracted from a cell. The reaction can involve a reaction with one or more components integrated in or coated on a bead structure, for example, a reagent-containing bead or ZipCoded® beads. For example, as described in Weiner et al., Introduction to SNPs: Discovery of Markers for Disease, BioTechniques, 32:S4-S13 (June 2002), which is incorporated herein in its entirety by reference.

Herein, the term nucleic acid sequence refers to any sequence of nucleotide bases, for example, a sequence held together by a sugar-phosphate backbone. The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 6 methyl-cytosine, uracil, thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O6-methylguanine, N6-methyladenine, O4-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide,” as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR2 or halogen groups, where each R is independently H, C1-C6 alkyl or C5-C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-anomeric nucleotides, 1′-anomeric nucleotides, 2′-4′- and 3′-4′-linked and other “locked” or “LNA”, bicyclic sugar modifications (see, e.g., PCT published application nos. WO 98/22489, WO 98/39352; and WO 99/14226). Exemplary LNA sugar analogs within a polynucleotide include, but are not limited to, the structures:

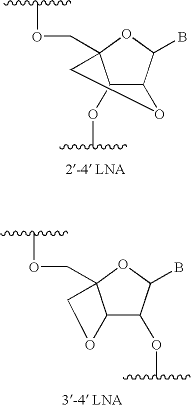

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N9-position of the nucleotide base. When the nucleotide base is pyrimidine, e.g. C, T or U, the pentose sugar is attached to the N1-position of the nucleotide base, except for pseudouridines, in which the pentose sugar is attached to the C5 position of the uracil nucleotide base (see, e.g., Kornberg and Baker, (1992) DNA Replication, 2nd Ed., Freeman, San Francisco, Calif.).

One or more of the pentose carbons of a nucleotide may be substituted with a phosphate ester having the formula:

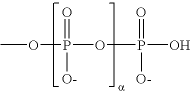

where α is an integer from 0 to 4.

In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. -thio-nucleotide 5′-triphosphates. For a review of nucleotide chemistry, see: Shabarova, Z. and Bogdanov, A. Advanced Organic Chemistry of Nucleic Acids, VCH, New York, 1994.

The term “nucleotide analog,” as used herein, refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of nucleotide “analog” are nucleotide analog monomers which can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone. Also included are intercalating nucleic acids (INAs, as described in Christensen and Pedersen, 2002), and AEGIS bases (Eragen, U.S. Pat. No. 5,432,272).

As used herein, the terms “polynucleotide”, “oligonucleotide”, and “nucleic acid” are used interchangeably and mean single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H+, NH4+, trialkylammonium, Mg2+, Na+ and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occurring nucleotides and nucleotide analogs. Nucleic acids typically range in size from a few monomeric units, e.g. 5-40 when they are sometimes referred to in the art as oligonucleotides, to several thousands of monomeric nucleotide units. Unless denoted otherwise, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Nucleic acids include, but are not limited to, genomic DNA, cDNA, hnRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below:

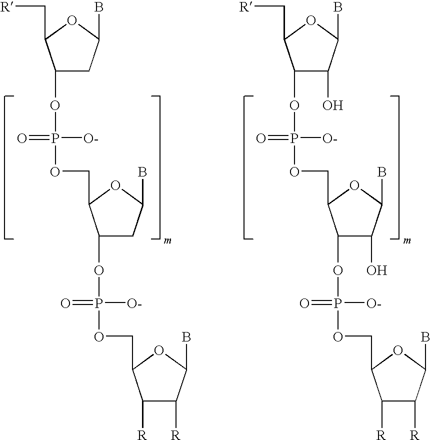

wherein each B is independently the base moiety of a nucleotide, e.g., a purine, a 7-deazapurine, a pyrimidine, or an analog nucleotide; each m defines the length of the respective nucleic acid and can range from zero to thousands, tens of thousands, or even more; each R is independently selected from the group comprising hydrogen, halogen, —R″, —OR″, and —NR″R″, where each R″ is independently (C1-C6) alkyl or (C5-C14) aryl, or two adjacent Rs are taken together to form a bond such that the ribose sugar is 2′,3′-didehydroribose; and each R′ is independently hydroxyl or

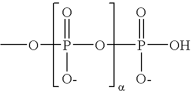

where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably and, as used herein, refer to a nucleic acid that contains at least one nucleotide analog and/or at least one phosphate ester analog and/or at least one pentose sugar analog. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C1C4 alkylphosphonate, e.g. methylphosphonate; (ii) phosphoramidate; (iii) C1C6 alkyl-phosphotriester; (iv) phosphorothioate; and (v) phosphorodithioate.

According to various embodiments, the cytometric system can comprise a cytometric sorting station. The cytometric sorting station can comprise any suitable sorting device capable of separating a biomolecule-containing material from a sample, for example, capable of separating a nucleic acid sequence-containing biological material from a sample containing various components. The nucleic acid sequence-containing biological material can be a mixed (heterogeneous) population of cells in a cell culture, cellular components such as nucleic acid sequences, or the like. The nucleic acid sequence-containing biological material sorting station can sort the biological material based on size, molecular weight, cell type, staining characteristics, reflectivity, antibody binding, electric charge, or the like. The cytometric sorting station can comprise a flow cytometry device, a cytofluorometry device, an ion-exchange chromatography column, an electrophoresis device, a surface modification device, an optoelectronic biomolecule separation device, an optoelectronic scanning device, or the like. Exemplary surface modification devices that can be used to sort biomolecules include those described by Lau in U.S. patent application Ser. No. 10/979,645 filed Nov. 1, 2004. Exemplary optoelectronic biomolecule devices that can be used include those described in U.S. Provisional Patent Application No. 60/692,528 filed Jun. 20, 2005. Exemplary optoelectronic scanning devices that can be used for biomolecule sorting include those described in U.S. Provisional Patent Application No. 60/731,123 filed Oct. 27, 2005. Each of the aforementioned patent applications is incorporated herein in its entirety by reference. In some embodiments, the sorting station can sort a single cell from a mixture, or a single type of cell from a mixture. The cytometric sorting station, can use motive forces, for example, electrostatic forces, mechanical movement, electric field gradients, vacuums, streams of air, or other motive forces as known to those of ordinary skill in the art, for sorting a nucleic acid sequence-containing component from a sample. Nucleic acid sequence containing components can comprise, for example, one or more cell types, or michondria. Cytometric sorting can be performed in a sterile environment or can be performed under non-sterile conditions.

The cytometric sorting station can comprise a flow cytometer, for example, of the type that uses electrostatic deflection of charged droplets to separate droplets or components, in a fashion similar to that used in ink-jet printers. Cells can be aspirated from a sample and ejected one by one from a nozzle in a stream of sheath fluid, for example, in a stream of phosphate-buffered saline (PBS), although any of a variety of ionized fluids can be used.

Most streams are unstable with respect to time and will eventually break up into droplets. It is possible to stabilize this break-off point by applying a stationary wave of vibration of known frequency and amplitude, to the stream. When cells are the nucleic acid sequence-containing biological material to be separated, the cells can be illuminated with a laser beam. As the cells are illuminated by the laser beam, scattered light and fluorescence signals can be generated. The signals can be interpreted by sort logic boards which can make decisions as to whether a particular cell is to be sorted or not, for example, according to user-defined criteria.

The distance between the laser illumination site and the break-off point is called the drop delay. If a cell of interest, that is, a cell of a type to be sorted, has been detected, the cytometric separation can wait until that cell has traveled to the break-off point before exposing the stream to a charge. Accordingly, as a droplet containing a cell of interest leaves a solid fluid stream it can be made to carry a charge, either positive or negative. At a further distance downstream, the charged droplet can be made to pass through two high voltage deflection plates where it will become attracted toward the plate of opposite polarity. It is therefore possible to sort two or more separate populations from the same sample. By applying different levels of charge to the left side or to the right side of the stream.

According to various embodiments, other methods of cytometric cell sorting can be used, for example, that comprise the use of compressed air rather than electric charge to separate cells from a cell stream. One such device is available from DakoCytomation of Fort Collins, Colo. under the registered tradename MOFLO Sorters.

According to various embodiments, the cytometric sorting station can comprise two or more retainment regions capable of retaining components of a sample of biological material sorted by the cytometric sorting station. The retainment regions can comprise a suitable beaker, cup, dish, tube, container, reservoir, well, chamber, capillary, or the like. The retainment regions can be, for example, wells in a fluid processing or fluid containment device, wells in a card-type fluid processing device, or wells in a microcard or microtiter plate, for example, formatted to include one, two, four, eight, 16, 24, 48, 96, 192, 384, 1536, 6144, or more wells.

According to some embodiments, the cytometric system can comprise a single movable electrode or multiple selectable electrodes, that can be used, for example, to thermal cycle the material contained in a retainment region. One or more retainment regions can comprise a conductive bottom surface.

According to various embodiments, the cytometric system can comprise a cell preparation station capable of isolating nucleic acid sequences from other components of a sample. The cell preparation station can comprise a device for separating nucleic acid sequences from other biological materials, for example, from other cell components of a cell from which the nucleic acid sequence originated. The cell preparation station can comprise a centrifuge device, a mechanical cell lysis device, a chemical cell lysis device, a DNA extraction device, or the like. Preparation reagents, as discussed below, can be added to or included with the preparation station.

The cell preparation station can include a device for lysing cells with any suitable method. Suitable methods can comprise, for example: thermal lysis, mechanical lysis, sonic lysis, chemical lysis, enzymatic lysis, combinations thereof, or the like methods. The preparation station can be capable of adjusting the pH of a biological material through the introduction of suitable acids, bases, or buffers. The preparation station can be capable of thermally adjusting, or maintaining the temperature of, a biological material. Cell poration devices, for example, electroporation devices, can be used instead such that lysing of the cell is not necessary. Other biomolecule extraction devices and methods can be used, for example, extraction using a hollow needle.

According to various embodiments, a nucleic acid sequence-containing component of a cell can be extracted from other components of the cell by any of a variety of methods. For example, the cell can first be lysed, for instance, by using enzymes such as e.g. proteinase K or lysozyme, by using detergents such as SDS, Brij, Triton X 100, Tween 10, and DOC, or by using chemicals such as sodium hydroxide, guanidine hydrochloride, and/or guanidine isothiocyanate. The resulting cell fragments can be separated or sedimented, for example, by centrifugation, and/or membrane filtration. Separation or sedimentation can be performed at, for example, atmospheric pressure, greater than atmospheric pressure (positive pressure), less than atmospheric pressure (vacuum), or any combination thereof. The supernatant can then be decanted or pipetted-off and subsequently purified by chromatography, or by extraction with either phenol or chloroform, followed by an alcohol precipitation. Centrifugation can be carried out, for example, in a conventional laboratory centrifuge, such as a Heraeus Biofuge GL, and can take from about 15 minutes to about two hours at a rate of from about 3,000 rpm to about 20,000 rpm, depending on the particular application and the particle sizes of the cell fragments.

According to some embodiments, the cell preparation station can comprise a filtering device. The filtering device can be an ion-exchange chromatography column of the type commonly used in the art, or it can be an ion-exchange material or column of the type described in U.S. patent application Ser. No. 10/414,179, filed Apr. 14, 2003, to Lau et al., which is incorporated herein in its entirety by reference.

According to various embodiments, the detection station of the system can comprise a nucleic acid sequence amplification station. The amplification station can comprise any suitable device capable of amplifying nucleic acids. The amplification station can be a polymerase chain reaction (PCR) device, a real time (RT) PCR device, a ligase chain reaction device, an isothermal amplification reaction device, or a signal amplification device such as a device that carries out an Invader® assay (available from Third Wave Technologies, Inc of Madison, Wis.). Although referred to herein as a nucleic acid sequence amplification station, it is to be understood that the station could carry out a signal amplification method such as an Invader® assay instead of actually amplifying or making replicates of a target nucleic acid sequence. Additional information about the Invader® assay, and methods and devices for carrying out such an assay, is described in U.S. Pat. No. 6,706,471 which is incorporated herein in its entirety by reference.

According to some embodiments, the nucleic acid sequence amplification station carries out a replication reaction. The station can comprise a nucleic acid synthesis device, for example, as taught in U.S. Pat. Nos. 4,683,195 (Mullis et al.), 4,683,202 (Mullis), or 4,965,188 (Mullis et al.), each of which is incorporated herein in its entirety by reference. In exemplary methods of amplification, one or more nucleic acid templates can be hybridized with smaller complementary “primer” nucleic acids in the presence of a thermostable DNA polymerase and deoxyribonucleoside triphosphates. Upon hybridization of a primer and a template to form a “primed template complex,” DNA polymerase can extend the primer in a template directed manner to yield a primer extension product. Primer extension products can then serve as templates for nucleic acid syntheses. Upon denaturation, the primer extension products can hybridize with primers to form primed template complexes that can serve as DNA polymerase substrates. Cycles of hybridization, primer extension, and denaturation can be repeated many times to exponentially increase the number of primer extension products.

According to various embodiments, the nucleic acid sequence amplification station can comprise a thermal cycling device. The cycles of hybridization, primer extension, and denaturation can be conducted by cycling the reactants through different temperatures with the thermal cycling device. The specific temperatures used can be based upon the desired base paring efficiency and can be deduced by those skilled in the art, based upon the base composition of the nucleic acid samples and primers.

According to various embodiments, a real-time PCR (RT PCR) reaction device can be used in the amplification station. The RT PCR device can be similar to a PCR reaction device except that one or more reactant, primer, interculator, or other “probe,” can be used that is labeled with a marker, for example, a fluorescent dye marker. Any suitable marker, such as, for example, a fluorophore, can be used. Fluorophores useful according to various embodiments can comprise those that can be coupled to organic molecules, particularly proteins and nucleic acids, and that can emit a detectable amount of radiation or light signal in response to excitation by an available excitation source. Suitable markers can include, for example, materials having fluorescent, phosphorescent, and/or other electromagnetic radiation emissions. Irradiation of the markers can cause them to emit light at respective frequencies depending on the type of marker used.

Labeled primers (probes) can further comprise a quenching molecule so that the probe undergoes fluorescence resonance energy transfer (FRET). FRET is a distance-dependent interaction between the electronic excited states of two dye molecules in which excitation is transferred from a donor molecule to an acceptor molecule without emission of a photon. The efficiency of FRET can be dependent on the inverse sixth power of the intermolecular separation, making it useful over distances comparable with the dimensions of biological macromolecules.

FRET type probes or primers can be used with a suitable polymerase. The polymerase can copy a complementary strand of nucleic acid and digest the probes. This digestion can disrupt the FRET and can allow the observance of the reporter dye with equipment know in the art. These observations can be used to track the progress of nucleic acid replication.

According to various embodiments, the nucleic acid sequence amplification station can comprise an ABI PRISM® 7900 HT device, an Applied Biosystems RT PCR System, an ABI PRISM® 7000 HT device, an ABI PRISM® 7300 HT device, or an ABI PRISM® 7500 HT device, all available from Applied Biosystems, Foster City, Calif., for example, to amplify and/or detect, a nucleic acid sequence, a gene sequence, or a single nucleotide polymorphism (SNP). A SNP can comprise a single deletion, addition, or a point mutation.

According to various embodiments, the nucleic acid sequence amplification station can comprise or be in operational communication with a device capable of sequencing an amplified nucleic acid sequence and/or subjecting an amplified nucleic acid sequence to a sequencing reaction. Suitable sequencing devices and/or sequencing reaction devices can comprise, but are not limited to, polyacrylamide gel sequencing devices, slab-gel sequencing devices, capillary electrophoretic sequencing devices, cycle sequencing devices, thermal-cycling sequencing devices, stepwise sequencing devices, ZMWG devices, shotgun sequencing devices, Sanger sequencing devices, devices employing the chain-terminating inhibition reaction method or the base specific chemical cleavage method, ZMWG stepwise sequencing devices, optical fluorescence sequencing devices, combinations thereof, or the like. One exemplary device for conducting nucleic acid sequencing is the ABI PRISM® 7900 HT Sequence Detection System available from Applied Biosystems of Foster City, Calif.

According to some embodiments, the cytometric system can comprise a nucleic acid sequencing station, for example, provided downstream of the nucleic acid sequence amplification station, or integrated with the sequence amplification system. Nucleic acid sequencing can be performed using, for example, Applied Biosystems 3730XL DNA analyzer available from Applied Biosystems, Foster City, Calif.

According to various embodiments, the cytometric system can comprise a detection station that can be integrated with, or separate from, the nucleic acid sequence amplification station. The detection station can comprise, for example, an electrophoresis device, a temperature gradient electrophoresis device, a capillary electrophoresis device, a chromatography column device, a mass spectrometry device, a nuclear magnetic resonance device, or the like. Exemplary detection devices can comprise the ABI 3730 or ABI 3730XL capillary electrophoresis DNA analyzer, both available from Applied Biosystems, Foster City, Calif. The detection system can comprise a device capable of focused resequencing, SNP detection, nucleic acid sequence detection, and/or gene expression detection.

If an electrophoresis detector is included in the cytometric system, the detector can comprise, for example, a channel-defining member such as an electrophoretic plate or capillary tube, wherein opposing ends of the channel-defining member can be placed in contact with corresponding electrodes connected to a power supply for generating an electric field across the member. The field can be used to cause analytes of a sample to migrate from a loading site for the member toward a detection site or detection zone of the member. The detection site or detection zone can encompass a zone along the member which can be irradiated by an irradiation source to excite markers, such as dye markers, used to label the analytes.

According to various embodiments, the cytometric system can comprise a conduit system that can be capable of directing a sample or sample component, for example, the nucleic acid sequence-containing component of a biological material, between the various stations of the cytometric system. The conduit system can comprise, for example, a single conduit connecting the sorting station and the amplification station. The conduit system can comprise multiple conduits connecting one or more of the sorting station, the amplification station, the preparation station, or the detection station, to one or more of the other stations. The conduit system can comprise a plurality of conduits connecting the stations in a variety of combinations. The individual conduits of the conduit system can comprise channels, tubes, capillaries, pipes, or the like.

According to various embodiments, the cytometric system can comprise one or more pumps. Exemplary pumps that can be used comprise, but are not limited to, liquid pumps, peristaltic pumps, piezo-electric pumps, hydraulic pumps, micro pumps, or the like. The pumps can be incorporated with, or be separate from, the conduit system. The pumps can be capable of inducing fluid flow along the conduit system. The pumps can be incorporated, for example, anywhere along the conduit system including between two or more of the various stations.

According to some embodiments, one or more of the stations of the cytometric system can be incorporated into a planar substrate such as a card or a chip. The planar substrate can be made of any suitable material including, glass, silicon, plastic, or the like. The planar substrate can be etched, cut, ground, molded, machined, or otherwise formed, so as to provide the conduit system and retainment regions of the one or more stations of the cytometric system.

According to various embodiments, the sequence amplification station can further comprise at least one platen. The platen can have a top surface and can be rotated. One or more retainment regions can be positioned upon the top surface of the platen, or in a device, for example, a microfluidic plate, disposed on the top surface of the platen, in such a way that the rotation of the platen will bring the retainment regions into contact with, for example, a thermal cycling apparatus.

According to some embodiments, during processing, an aliquot of a biological material can be separated from another aliquot of the biological material or can be separated from a different biological material, in a single region, by, for example, an intervening amount of an immiscible fluid. For example, after a target cell population has been sorted or separated by a flow cytometric separation from a biological sample, the sorted cell population can be divided into separate aliquots or can be separated from other portions of the biological sample, using an immiscible fluid, for example, using mineral oil or silicon oil.

According to some embodiments, a biological material, after sorting or separating from a biological sample, can be processed, for example, prepared, amplified, purified, sequenced, or the like, and can likewise be separated from other aliquots of the same processed material or from other different processed materials, by an immiscible fluid. An appropriate immiscible fluid injection unit and immiscible fluid source can be provided in fluid communication with one or more sample transfer conduits, and can be adapted or controlled to inject appropriate isolating aliquots of the immiscible fluid.

According to various embodiments the cytometric system can comprise a control unit. The control unit can be operatively connected to one or more of the sorting station, the amplification station, a preparation station, a detection station, pumps, and valves. Operatively connected can mean one or more of electrically connected, hydraulically connected, mechanically connected, optically connected, any combination of such connections, and the like. The control unit can comprise a computer system. The computer system can comprise a processor, ram, rom, and can have sufficient processing power to instruct the sorting station, the amplification station, and any other stations or devices that are included in the system. The control unit can comprise information input and output devices. The control unit can comprise buttons, keys, dials, or the like, and the output devices can be monitors, gages, readouts, or the like.

According to various embodiments the cytometric system can comprise a housing. The housing can comprise any suitable material, for example, one or more of metal, plastic, glass, composite material, any combination thereof, and the like. The housing can surround and protect one or more of the individual stations, and/or the conduit system, of the cytometric system. The housing can enable materials to be introduced into the stations and/or moved between the stations. The control unit can be positioned on the housing and function as an interface.

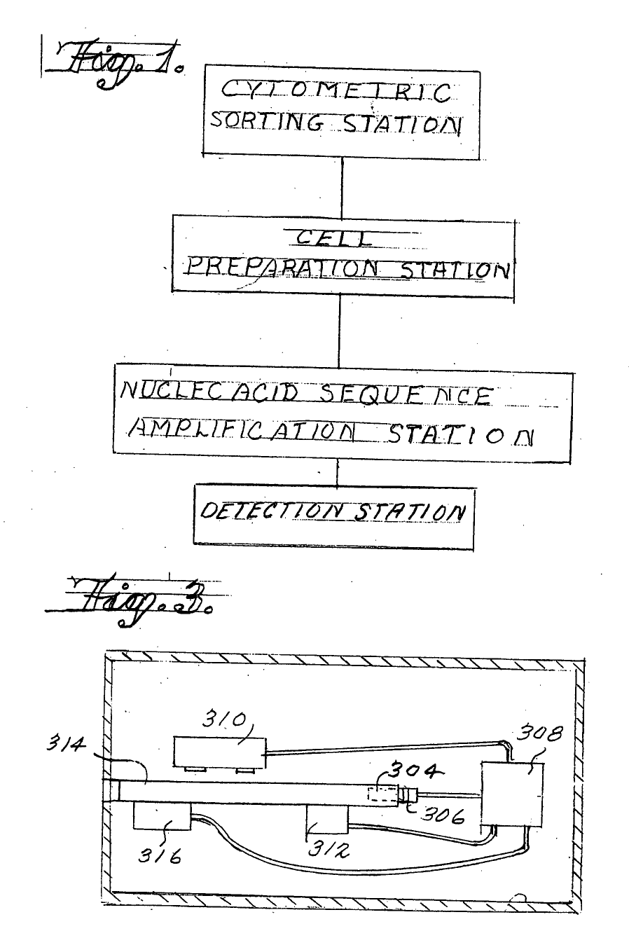

With reference to the drawings, FIG. 1 is a flow diagram detailing the interconnection of components according to various embodiments of the present teachings. As seen in FIG. 1, the system can comprise a cytometric sorting station for separating biological materials based on characteristic differences. The cytometric sorting station can be connected to a cell preparation station. The cell preparation station can prepare cellular material for nucleic acid amplification reactions. Preparation can include lysing and/or centrifugation.

The cell preparation station can be connected to a nucleic acid sequence amplification station. The nucleic acid sequence amplification station can provide conditions suitable for nucleic acid amplification reactions. Nucleic acids can be analyzed in a connected detection station. The detection station can comprise a capillary electrophoresis device, or the like. In some embodiments, proteins can be sorted and detected instead of nucleic acids.

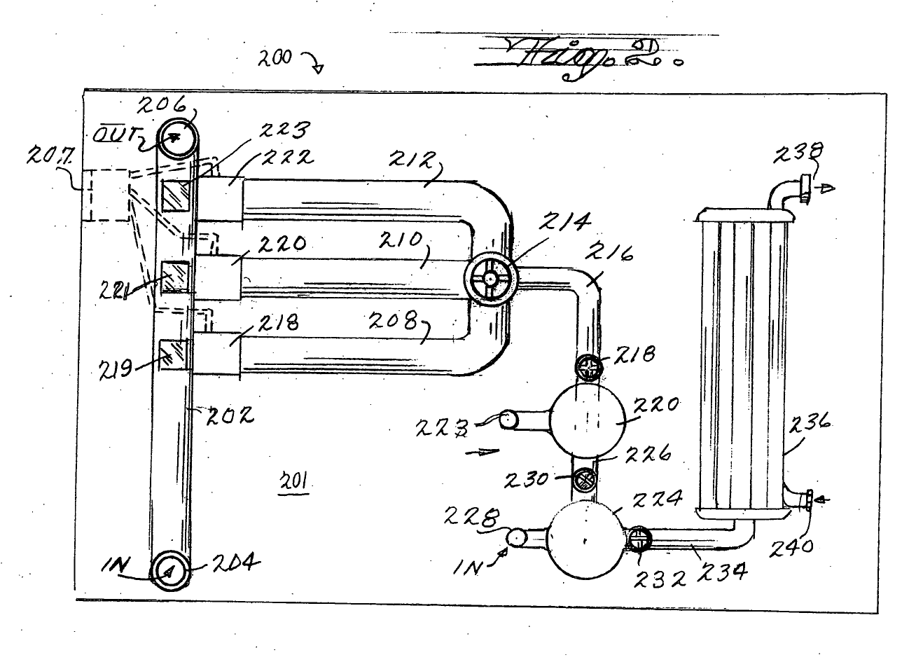

FIG. 2 is a perspective view of a cytometric system according to some embodiments of the present teachings, all in a card-type device, for example, a microfluidic plate. As seen in FIG. 2, the cytometric system 200 can comprise a conduit 202 including an entry port 204 and an exit port 206, and can be defined on or in a planar substrate such as a chip 201. A biologic sample can be placed into the entry port 204. From there it can be drawn by suction of otherwise towards exit port 206 by a pump attached thereto (not shown). One or more detection windows 219, 221, 223 may be present adjacent or above the conduit 202. One or more transfer conduits 208, 210, 212, each having first and second ends, can be disposed so as to provide a fluid communication between the conduit 202 and a valve 214, or the valve 214 can otherwise selectively control the flow of a material from one of the transfer conduits 208, 210, 212 into conduit 216. Instead of a valve, or in addition to the valve, a diverter can be used, for example, a controllable baffle flow diverter. One or more pumps 218, 220, 222 can be positioned in or adjacent the first end of one or more of conduits 208, 210, 212. Each pump 218, 220, 222, can be connected to an external electrical connector or interface 207.

The conduits 202, 208, 210, 212, the pumps 218, 220, 222, and the valve 214 can together comprise a cytometric sorting station. The conduit 216 can provide a fluid communication between the valve 214 and a preparation chamber 220. A second valve 218 can control access to the preparation chamber 220. The preparation chamber 220 can comprise an input port 223 through which preparation reagents can be added to the preparation chamber 220. The preparation chamber 220 can function as a preparation station for preparing biological materials for biological reactions such as nucleic acid amplification and or sequencing. The chamber 220 can comprise a centrifuge/lysis chamber, or access to a centrifuge/lysis chamber. Alternatively, preparation and amplification can occur in a single chamber.

The preparation chamber 220 can be fluidly connected to an amplification chamber 224 by a conduit 226. A valve 230 in conduit 226 can control access to the amplification chamber 224. An input port 228 can provide access to the amplification chamber 224 for the introduction of reagents suitable for nucleic acid amplification and the like, thereto. The chamber 224 can be positioned adjacent to a thermal cycling device (not shown). A thermal cycling device can allow for temperature cycling-dependent nucleic acid amplification reactions to be conducted. The combination of the preparation chamber 220, the amplification chamber 224, and the thermo cycling device 310, can be considered a nucleic acid sequence amplification station. The system can comprise one or more sequencing chambers provided, for example, downstream of the amplification chamber.

The chamber 224 can be fluidly connected to a capillary channel electrophoresis device 236. The capillary channel electrophoresis device 236 can be fluidly connected to two ports 238 and 240. The ports 238 and 240 can function to provide access for a suitable electrolyte and electrical current to and from the capillary channel electrophoresis device 236. The capillary channel electrophoresis device 236, with or without a detection block (not shown), can be considered a detection station.

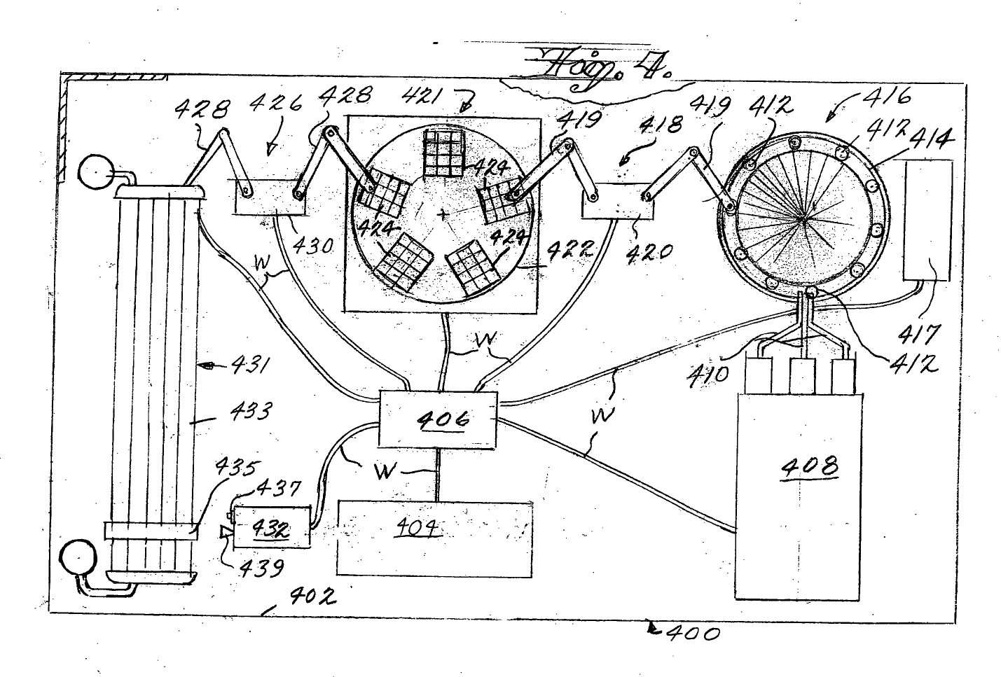

FIG. 3 is a sectional view according to various embodiments of the present teachings. In FIG. 3, a cytometric system 300 is shown with a chip 314 positioned inside a housing 302 with an electrical connector 304 on the chip 314 in electrical contact with an electrical lead 306 inside the housing. The electrical lead 306 can provide an operative connection with a processing unit 308. The chip 314 can comprise the system 200 illustrated in FIG. 2.

The processing unit 308 can be operatively connected to a detection block 310. The processing unit 308 can electrically connect to, and control the operation of one or more pumps 312, which can control the flow of fluid through the chip 314. The detection block 310 can comprise an excitation source, for example, a laser or photodiode, and a detection device, for example, a camera, a photodetector, a CMOS detector, or the like. The detection block 310 can function to illuminate a labeled or marked biological material, and identify the illuminated biological material. The detection block 310 can also be positioned to excite tagged biological molecules such as nucleic acids that are separated in a capillary electrophoresis detection device. Excited fluorogenic nucleic acid sequences can be detected by the detection block 310.

A thermal cycling device 316 can be provided to heat and cool the chip 314 sufficiently to promote temperature-dependent nucleotide amplification reactions. The thermal cycling device 316 can be operatively connected to a processing unit, such that the processing temperature of the chip 314 can be regulated.

FIG. 4 is a top plan view of a system according to various embodiments of the present teachings. The cytometric system 400 can comprise a housing 402 into which a control unit 404 can be incorporated. The control unit 404 can be operatively connected to a processing unit 406. The control unit 404 can serve as an interface between a user and the processing unit 406.

The processing unit 406 can be operatively connected to a cytometric sorting station 408. The cytometric sorting station 408 can be configured to sort nucleic acid sequence-containing biological materials from a biological sample, into one or more channels 410 that can provide a fluid communication with one of several retainment regions 412. The retainment regions 412 can hold or selectively release nucleic acid sequencing-containing biological materials sorted by the cytometric sorting station 408. The retainment regions can comprise disposable vials or containers.

The preparation station 416 can comprise a centrifuge 414 or other device suitable for the preparation of biological material for nucleic acid amplification. The cytometric system 400 can comprise a reagent loading device 417. The reagent loading device 417 can load one or more reagents, for example, one or more reagents suitable for lysing cells and/or separating DNA from the materials present in the lysate, and/or for cleaving cellular DNA into manageable fragments. Reagents can be loaded before and/or after target cells are loaded into the centrifuge 414. Reagents suitable for cleaving DNA can comprise, for example, one or more of endonucleases and restriction endonucleases. A positionable conduit system 418 can provide a fluid communication between the preparation station 416 and an amplification station 421. The conduit system 418 can comprise a pumping block 420 to facilitate fluid transfer to the amplification station 421, and robotic, positionable conduits 419. The amplification station 421 can comprise a rotatable platen 422, at least one multi-well plate 424, and a thermal cycling device (not shown). The conduit system 418 can be positionable so as to direct fluid and/or samples from the preparation station 416 into individual wells in the multi-well plates 424. The conduit system 418 can be robotically controlled.

A second conduit system 426 can be positioned so as to provide a fluid communication between the amplification station 421 and an electrophoretic detection device 431. A pumping block 430 can be incorporated into the conduit system 426 to facilitate fluid transfer along robotic, positionable conduits 428. The conduit system 426 can be operatively connected to the control unit 406. The electrophoresis device 431 can comprise, for example, one or more electrophoretic separation channels 433.

The cytometric system 400 can further comprise a detection block 432. The detection block 432 can comprise, for example, an LED excitation source 437 and a CCD detector 439 arranged to excite and detect, respectively, fluorescent dyes passing through a detection zone underneath a detection window 435. Detection windows, excitation sources, and detectors that can be used include those described in U.S. patent applications Ser. Nos. 10/205,028, 10/887,486, and 10/887,528, all of which are incorporated herein, in their entireties, by reference, as described above or as involved in fabricating such a device. The capillary electrophoresis device 431 and the detection block 432 can be operatively connected to the control unit 406.

While the present teachings are described in conjunction with various embodiments, it is not intended that the present teachings be limited to such embodiments. On the contrary, the present teachings encompass various alternatives and modifications as will be appreciated by those of skill in the art.

1. A system comprising:
a sorting station adapted to sort a nucleic acid sequence-containing biological material from a nucleic acid sequence-containing biological sample;
a detection station adapted to amplify, detect, or amplify and detect, at least a portion of a nucleic acid sequence to form a product; and
a conduit system capable of directing a nucleic acid sequence-containing biological material from the sorting station to the detection station, and comprising at least one valve or diverter.

2. The system of claim 1, wherein the sorting station is adapted to sort the nucleic acid sequence-containing biological material by at least one of size, molecular weight, cell type, staining characteristics, reflectivity, antibody binding, electric charge, and a combination thereof.

3. The system of claim 1, wherein the sorting station comprises a flow cytometry device.

4. The system of claim 1, wherein the conduit system comprises at least one pump.

5. (canceled)

6. The system of claim 1, wherein the conduit system comprises at least one valved conduit, wherein the at least one valved conduit is simultaneously in fluid communication with both the sorting station and the detection station.

7. The system of claim 1, wherein the detection station comprises a thermal cycler.

8. The system of claim 1, further comprising at least two retainment regions, each adapted to retain a respective different nucleic acid sequence-containing biological material sorted by the sorting station, wherein the conduit system is adapted to direct nucleic acid sequence-containing biological material from each of the at least two retainment regions to the detection station.

9. The system of claim 8, wherein the detection station comprises an amplification station comprising reagents to enable polymerase chain reaction of a nucleic acid sequence, upon thermal cycling.

10.-11. (canceled)

12. The system of claim 1, further comprising a control unit operatively connected to the sorting station and to the detection station.

13. (canceled)

14. The system of claim 1, further comprising a preparation station capable of isolating a nucleic acid sequence from a nucleic acid sequence-containing biological material, wherein the conduit system is adapted to direct a nucleic acid sequence-containing biological material from the sorting station to the preparation station and adapted to direct an isolated nucleic acid sequence from the preparation station to the detection station.

15. (canceled)

16. The system of claim 1, wherein the detection station comprises an amplification station and a detector, and wherein the conduit system is capable of directing a product from the amplification station to the detector.

17. (canceled)

18. A system comprising:
a housing;
a sorting station disposed within the housing and adapted to sort a nucleic acid sequence-containing biological material from a mixture of biological materials;
an amplification station disposed within the housing and adapted to amplify, detect, or amplify and detect, at least one nucleic acid sequence; and
a conduit system disposed within the housing and adapted to direct a nucleic acid sequence-containing biological material from the sorting station to the amplification station.

19. The system of claim 18, further including;
at least two retainment regions adapted to retain a nucleic acid sequence-containing biological material sorted by the sorting station, wherein the conduit system is adapted to directing the biological material from each of the at least two retainment regions to the amplification station.

20. The system of claim 19, further including;
a preparation station disposed within the housing, wherein the conduit system is adapted to direct the nucleic acid sequence-containing biological material from at least one of the retainment regions to the preparation station.

21. The system of claim 18, wherein the conduit system is adapted to transfer the first nucleic acid sequence-containing biological material from the sorting station to the amplification station.

22. The system of claim 18, further including;
a detection station disposed within the housing, wherein the conduit system is adapted to transfer the biological material from the amplification station to the detection station.

23. (canceled)

24. The system of claim 23, further including:
a control panel operatively connected to the housing; and
a control system operatively connected to the control panel, wherein the control system is operatively connected to the sorting station, the amplification station, the preparation station, the detection station, and the plurality of pumps.

25. The system of claim 18, wherein the amplification station comprises a multi-well plate.

26. The system of claim 18, wherein the conduit system comprises at least one conduit, wherein at least one conduit is simultaneously in fluid communication with both the sorting station and the amplification station.

27. The system of claim 22, wherein the amplification station is adapted to sequence a nucleic acid sequence.

28. (canceled)

29. A method of analyzing a nucleic acid sequence-containing biological material, comprising:
introducing a nucleic acid sequence-containing biological sample into a sorting station;
sorting the nucleic acid sequence-containing biological material from the biological sample;
moving the nucleic acid sequence-containing biological material through a conduit system to an amplification station; and
amplifying, detecting, or amplifying and detecting, nucleic acids present in the nucleic acid sequence-containing biological material.

30. The method of claim 29, further comprising:
moving the nucleic acid sequence-containing biological material through a conduit system from the sorting station to a preparation station;
preparing the nucleic acid sequence-containing biological material for nucleic acid amplification;
moving the nucleic acids through the conduit system from the amplification station to a detection station; and
detecting the nucleic acids.

31.-35. (canceled)
